(19) United States
(12) Patent Application Publication
(10) Pub. No.: US 2008/0123810 A1
(43) Pub. Date: May 29, 2008

(54) BOLUS MATERIALS FOR RADIATION THERAPY AND METHODS OF MAKING AND USING THE SAME

(76) Inventors: John P. Kirkpatrick, Chapel Hill, NC (US); Farokh R. Demehri, Gainsville, FL (US); Sara E. Johnston, Augusta, GA (US); Andrew M. Stalnecker, New York, NY (US); Tabitha M. Cooney, Tampa, FL (US)

Correspondence Address: JENKINS, WILSON, TAYLOR & HUNT, P. A., 3100 TOWER BLVD., Suite 1200, DURHAM, NC, 27707, US

(21) Appl. No.: 11/926,829

(22) Filed: Oct. 29, 2007

Related U.S. Application Data
(60) Provisional application No. 60/856,699, filed on Nov. 3, 2006.

(51) Int. Cl. A61N 5/00; A61B 6/00

(52) U.S. Cl. 378/65; 424/78.06

(57) ABSTRACT

Bolus materials for use in radiation therapy methods of making such bolus material and methods of providing radiation therapy using such bolus material are provided. The bolus materials can be an oil gel that includes at least one thermoplastic elastomer and an oily substance. The bolus materials are transparent and can have a maximum strain of at least 50%, a Young's Modulus of less than about 0.1 GPa, and a hardness less than about 90 on the Shore A scale.

### RELATED APPLICATIONS

The presently disclosed subject matter claims the benefit of U.S. Provisional Patent Application Ser. No. 60/856,699, filed Nov. 3, 2006; the disclosure of which is incorporated herein by reference in its entirety.

### TECHNICAL FIELD

The subject matter described herein relates generally to bolus materials and methods for using the bolus materials in radiation therapy, permitting higher surface doses in megavoltage photon treatments and shallower treatment depths in electron therapy. More particularly, the subject matter disclosed herein relates to bolus materials that offer clarity, stability and aesthetic properties for better and easier use within radiation therapy.

### BACKGROUND

Radiation therapy is used to treat diseases with the use of beams of either high-energy particles or waves. The most common radiation treatment used is external radiation from a machine outside of the patient's body. The machine is usually a linear accelerator. External beam radiation uses a variety of energy sources, including photons (X-rays or gamma rays), and particle beams (electrons, protons, neutrons). The type of energy used in the radiation therapy depends on what is best suited for treatment of a given patient.

During treatment, the radiation beam deposits energy that either injures or destroys the cells in the area being treated. The major effect of the radiation is DNA breakage, thereby destroying the genetic material that controls how the cells grow and divide. Since cells are more vulnerable to damage when they are dividing, and cancer cells divide more rapidly than their healthier counterparts, cancer cells have heightened susceptibility to radiation. Normal cells can thus recover from the effects of radiation more easily than cancer cells can. Although both types are damaged by radiation, the goal of treatment is to damage as few normal, healthy cells as possible. Treatment is staggered over several weeks to allow for the repair of injured, normal cells to help spare non-cancerous cells. During radiation therapy, patients may receive radiation five days a week over a period of one to eight weeks, with each session lasting about fifteen to thirty minutes. Radiation therapy delivers strong enough doses to destroy the cancer cells, while still sparing normal tissue from excessive radiation.

In some instances, radiation therapy may be the only viable treatment option. In other situations, radiation therapy could also be used in conjunction with surgery and/or chemotherapy. Periods of time when radiation therapy can be used include before surgery, to shrink a tumor as much as possible, during surgery, to direct radiation directly at a tumor, after surgery, to stop the growth of remaining cancer cells, and even to decrease pain or other symptoms associated with tumors.

As stated previously, external beam radiation can use a variety of energy sources, but most patients are treated with megavoltage photons. These forms of energy are popular due to skin-sparing properties, penetration and beam uniformity. For radiation involving megavoltage photons, the absorbed skin dosage is low (ranging from about 12% to about 17% of the maximum dose), and does not reach a maximum until one to four centimeters below the surface of the skin. The ability of this form of radiation therapy to spare the skin is very useful for many types of cancer, but problematic for treatment of superficial lesions at or near the skin surface.

To treat lesions on or close to the skin surface, bolus material can be placed over the skin region undergoing radiation therapy to increase radiation dosage at the skin surface. The bolus material can be a soft, rubbery tissue equivalent material placed in direct contact with the patient's skin surface. The bolus material increases the radiation dosage to the patient's external surface by providing scattering of the beam and build-up of the radiation dose prior to the beam's entry into the skin. As a result, the radiation beam deposits the maximum radiation dosage at or near the skin surface, rather than penetrating the skin and delivering the maximum dosage several centimeters below the skin surface, as the radiation beam would normally do.

There are several bolus products currently available, each with its own advantages and disadvantages, as described below in Table 1. These products all share Food and Drug Administration (FDA) approval and electron-absorption characteristics that are comparable to that of water. The products range from pre-formed gel sheets that are draped over the target area to materials that are molded onto the target area where they solidify. Examples of the pre-formed gel sheets include SUPERFLAB bolus material and ELSATO-GEL bolus that are both sold by Radiation Products Design, Inc. of Albertville, Minn. Examples of materials that are molded onto the target area where they solidify include SUPER STUFF and Beeswax both of which are sold by Radiation Products Design, Inc. of Albertville, Minn., and AQUAPLAST RT Custom Bolus manufactured and distributed by WFR/Aquaplast Corporation of Wycoff, N.J. A radiation oncologist may even choose to simply use saline-soaked gauze as a bolus for use during radiation therapy. Basically, a bolus need only form a uniform layer above the target area and have electron-absorption properties similar to that of tissue (or water).

TABLE 1
Current Bolus Technology

| Material | Tradename | Composition | Transparency | Properties | Safety |
|---|---|---|---|---|---|
| Gels | SUPERFLAB | vinyl plastic w/ | semi-transparent | Ready-made, flexible | Made of |
| | (the most widely used | di-isodecyl phthalate, | | gel sheets, uniform | materials FDA |
| | product on the | water based gels with | | thickness, conform | approved for |
| | market), SUPER- | acrylic polymer | | to skin. | human contact, |
| | FLEX, ELASTO-GEL | | | D = 1.02 g/cm3 | but can corrode |
| | | | | | plastic surfaces. |
| Moldable | SUPER STUFF, | solid, moldable bolus, | Clear upon | Comes as powders | FDA approved |
| boluses | AQUAPLAST | hydrophilic organic | application (hot), | that are mixed with | |
| | RT Custom Bolus, | polymer | opaque after | water or pellets that | |
| | and ADAPT-IT | | hardening (cool) | are molded. | |
| | Thermoplastic Pellets | | | Conforms well to | |
| | | | | steep curves, no | |
| | | | | flow/creep after | |
| | | | | drying. | |
| | | | | D = 1.02 g/cm3 | |
| Waxes | Beeswax | Natural hydrocarbon | Opaque | Cheap, natural wax | FDA approved |
| | Paraffin Wax | wax | | products that are | |
| | | | | molded to skin; not | |
| | | | | flexible. | |

Despite the wide array of bolus materials on the market, all of the current bolus products are lacking in that they are not clearly transparent. At best, some of these products can be considered translucent. Thus, no matter which bolus material a radiation oncologist chooses, the positioning of the radiation beam onto a patient's lesion will prove difficult, as the target area becomes obscured by the bolus material. During the course of a radiation therapy session, the precise alignment of the lesion under the radiation beam becomes increasingly uncertain. Even for bolus materials that have some level of translucency, their opacity often makes the underlying lesion and/or the guiding marks placed on the patient by the radiation oncologist hard to view through the bolus material. Furthermore, many of the existing bolus products are volatile or carry excessive stiffness or odor.

Therefore, a need exists for a transparent bolus material that would assuage a radiation oncologist's concerns with regard to correct placement of the radiation field with respect to the target tissue. Such a novel bolus could be placed over the target area by the radiation oncologist, who could then visually monitor the radiation field placement in relationship to the target lesion before and during a treatment session without having to remove the bolus, thus, ensuring that the entire target lesion, and only the target lesion, will be irradiated.

### SUMMARY

In accordance with this disclosure, transparent bolus materials, methods for making transparent bolus material, and methods of using transparent bolus material in radiation therapy are described that can be use to address oncologist's concerns with regard to correct placement of the target tissue. It is, thus, an object of the presently disclosed subject matter to provide novel transparent bolus materials, methods for making transparent bolus material, and methods of using transparent bolus material in radiation therapy.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 illustrates an embodiment of a bolus material that can be used in radiation therapy according to the present subject matter;

FIGS. 2A and 2B illustrate an embodiment of styrenic block copolymers that can be used in embodiments of a bolus material for use in radiation therapy according to the present subject matter;

FIG. 3 is a flow chart illustrating steps of an embodiment of a method to produce a bolus material that can be used in radiation therapy according to the present subject matter; and

FIG. 4 is a flow chart illustrating steps of an embodiment of a method of using an embodiment of a bolus material in radiation therapy according to the present subject matter.

### DETAILED DESCRIPTION

Reference will now be made in detail to the description of the present subject matter, one or more examples of which are shown in the figures. Each example is provided to explain the subject matter and not as a limitation. In fact, features illustrated or described as part of one embodiment can be used in another embodiment to yield still a further embodiment. It is intended that the present subject matter cover such modifications and variations.

As used herein, “bolus material,” “bolus,” or “bolus products” mean a soft, rubbery material that can serve as a tissue equivalent material that can be placed in direct contact with the patient's skin surface for improving dosage levels of radiation on and immediately below the skin surface.

As used herein, “clearly transparent” means that the material to which “clearly transparent” refers, such as a bolus material, is sufficiently transparent that a person having normal vision, i.e., 20/20 vision, can read black typing in 12 point font through at least 1 cm of the referenced material.

As used herein, “oil gel” means a blend of at least one oily substance and at least one gelling agent that when mixed together creates a gelatinous material.

As used herein, “oily substance” means an oil or oligomer that when mixed with a gelling agent can create an oil gel.

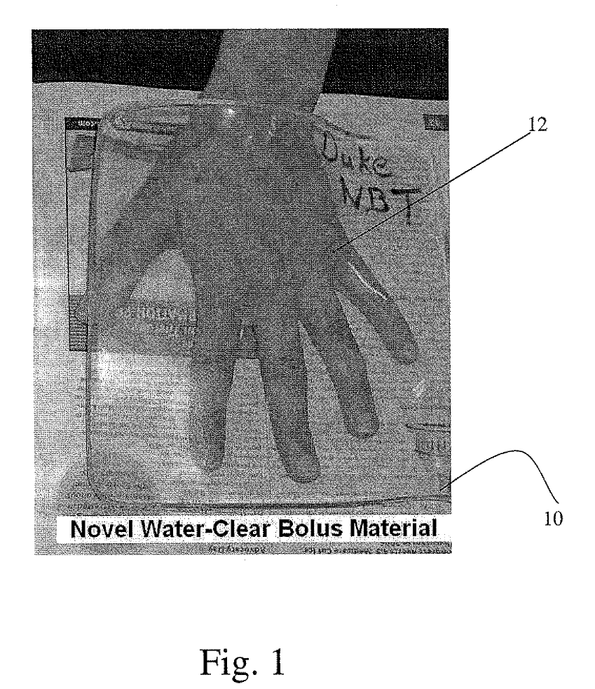

FIG. 1 is a photograph of a bolus material 10 draped over a hand 12. As can be seen from the photograph, the hand 12 can clearly been seen through bolus material 10. Bolus material 10 can be used by a radiation oncologist when treating skin lesions at or near the skin surface of a patient during radiation therapy. Bolus material 10 can be placed over the skin region undergoing radiation therapy to increase the radiation dosage at or near the skin surface. Bolus material 10 was created by selecting the appropriate components and mixing them in the correct proportions in order to achieve optimal material properties. As can be seen, the bolus material 10 is clearly transparent to permit the radiation oncologist to clearly see the portion of the patient that needs to be treated to identify where to focus the treatment. As seen in the FIG. 1, newsprint could be read through, for example, 2 cm of the bolus material. However, bolus material 10 can range in thickness from about 0.5 cm to about 5.5 cm and still provide a transparency that allows clear visibility therethrough. The thickness of the bolus material used in radiation therapy can vary depending on factors such as the type of radiation being used as well as the energy level of the radiation. With transparent bolus material 10, the radiation oncologist can more easily maximize the treatment by positioning the focus of the treatment by sight to ensure accuracy of treatment, if necessary.

Bolus material 10 also exhibits other physical properties that are beneficial when used in radiation therapy. Bolus material 10 is generally odorless, non-tacky, non-adherent, and is composed of materials approved by the FDA for human skin contact. For example, the bolus material 10 can have a Young's Modulus of less than about 0.1 GPa, hardness less than about 90 on the Shore A scale to provide adequate drape, and ability to withstand about 50% strain. Additionally, bolus material 10 can exhibit stability within a temperature range of about 40° F. to about 125° F.

Furthermore, bolus material 10 can also have tissue-equivalent dosimetric properties. Radiation dosimetry is the calculation of absorbed dose in matter and tissue resulting from the exposure to ionizing radiation. Thus, bolus material 10 can exhibit properties that are similar to that of human tissue in increasing dosage of radiation through a certain thickness by providing extra scattering or energy degradation of the beam. These dosimetric properties can be measured. For example, such dosimetric properties can be measured by computed tomography (CT) numbers, calculated mean Z or effective atomic number Z, and electron density.

Bolus material 10 is an oil gel which is primarily blends of oily substances and gelling agents. The types of oily substances and the types of gelling agents used in bolus material 10 produce the characteristics of bolus material 10 described above. Thermoplastic elastomers (TPE) can be used for the gelling agent. Non-limiting examples of such TPEs are suitable styrenic block copolymers. Examples of such suitable styrenic block copolymers include second generation styrenic block copolymers with a hydrogenated mid block of styrene-ethylene/butylene-styrene or styrene-ethylene/propylene-styrene. Second generation styrenic block copolymers with a hydrogenated mid block of styrene-ethylene/butylene-styrene or styrene-ethylenelpropylene-styrene as gelling agents can provide characteristics such as transparency, stability, and softness. Further, specific forms of these second generation styrenic block copolymers with a hydrogenated midblock of styrene-ethylene/butylene-styrene or styrene-ethylene/propylene-styrene have FDA approval for human skin contact.

For example, the TPE polymer can be or can include a translucent, linear triblock copolymer based on styrene and ethylene/butylene with a Styrene/Rubber ratio of about 30/70. For example, such a copolymer can comprise between about 10% and about 20% of the oil gel, for instance, between about 15% and about 17% of the oil gel. Another example of the second generation styrenic block copolymers with a hydrogenated midblock of styrene-ethylene/butylene-styrene or styrene-ethylene/propylene-styrene includes a clear, linear copolymer based on styrene and ethylene/butylene with a polystyrene content of about 33%. A further example includes a clear, linear triblock copolymer based on styrene and ethylene/butylene with a polystyrene content of about 30%. Such styrenic block copolymers can also by used in combination in the oil gel that comprises the bolus material.

The oily substances that can be used in the oil gel of bolus material 10 can be any suitable oily substance that when mixed with the gelling agent will provide a clearly transparent, odorless material. Non-limiting examples of the oily substances used in bolus material 10 include oils, such as mineral oils and naphthenic oils, and oligomers, such as polyterpene oligomers and polybutene oligomers. The oily substance can comprise a majority of the oil gel blend. Further, the above listed oily substances are compatible with the block copolymer, since the polystyrene segments of the suitable styrenic block copolymer can become solvated into the oily substances.

To achieve tissue-equivalent properties, a density can be obtained that is generally equivalent to water. Thus, for example, the density of bolus material 10 can be about 1 g/cm³. Alternatively, the thickness of the bolus material can be adjusted to obtain the desired tissue-equivalent properties. A bolus material 10 of an oil gel comprising mineral oil and suitable styrenic block copolymer can have a density less than about 1 g/cm³. For example, the oil gel comprising mineral oil and suitable styrenic block copolymer can have a density of between about 0.86 g/cm³ and about 0.91 g/cm³. Thus, such a bolus material may provide the desired tissue-equivalent dosimetric properties based on a ratio of thickness other than one to one. For example, computed tomography numbers of these materials ranged from about 130 HU to about 160 HU. Calculated mean Z can be 5.4 and electron density for such materials can be 3.05×10²³ e⁻/cm³ compared to 7.42 and 3.34×10²³ e⁻/cm³ for water, respectively. Thus, the current formulations would need to be utilized at slightly greater thicknesses to achieve build-up equal to water-equivalent materials. To compensate, the thickness of the bolus material 10 of the oil gel can be increased by a certain amount or percentage to obtain a suitable dosimetry that is generally equivalent to that of human tissue.

Alternatively, the bolus material 10 of the oil gel can further include a filler that can increase the density of bolus material 10 to a density level closer to that of water to achieve desired tissue-equivalent dosimetric properties. Thus, for example, the density of bolus material 10 of the oil gel can be brought to about 1 g/cm³, Since the oil gel described above generally has a density below about 1.0 g/cm³, a filler with a density higher than that of water can allow the density of bolus material 10 to average out to about 1.0 g/cm³. The filler should not interfere with transparency or the softness, strength, and low tack, of bolus material 10 to a point that bolus material 10 is not clearly transparent or the softness, strength, and tackiness render it useless for radiation therapy. The type and amount of filler used can vary depending on the amount and density of the oil and gelling agent. However, a filler is not needed to create a bolus material that provides desirable drape, clarity, strength, tack, odor, and tissue-equivalent dosimetric properties that can be used in radiation therapy.

The gelling agent and oil components that can comprise bolus material 10 are described in more detail below.

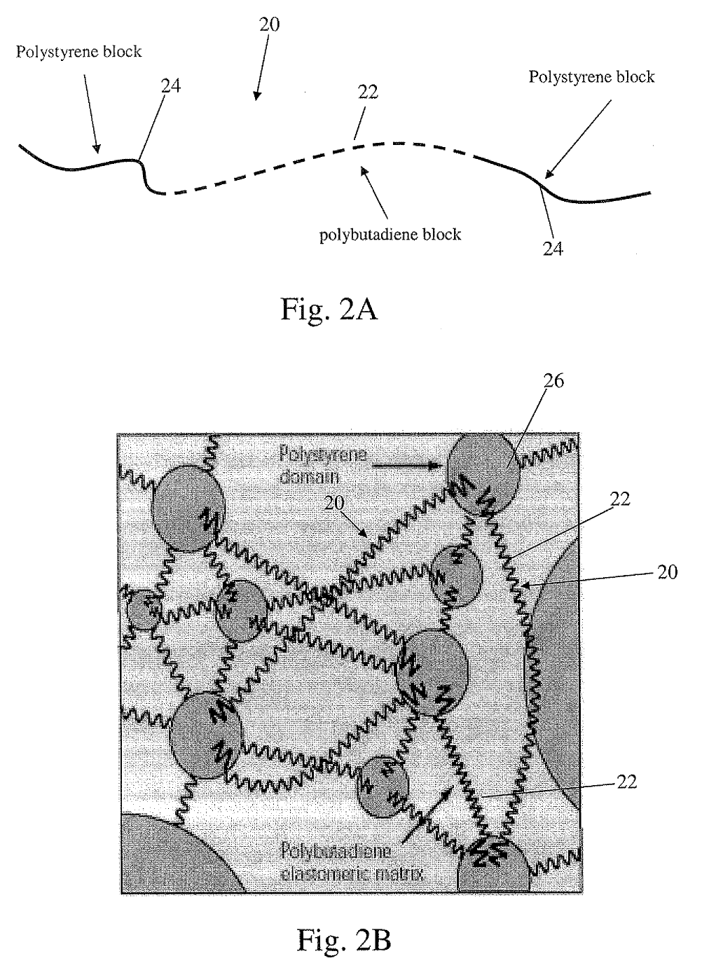

As stated above, the gelling agent can be TPEs. TPEs are polymeric materials that demonstrate both elastomeric (rubbery) and thermoplastic properties at room temperature. They are thermoplastic due to the ability to liquefy at higher temperatures and harden when cooled. A suitable TPE includes second generation styrenic block copolymers with a hydrogenated midblock of styrene-ethylene/butylene-styrene or styrene-ethylene/propylene-styrene; a non-limiting example of which is KRATON G polymers manufactured and sold by Kraton Polymers, LLC, of Houston, Tex. KRATON G polymers are part of a class of TPEs known as styrenic block copolymers. Another non-limiting example of a hydrogenated rubber styrene-ethylene/butylene-styrene that can be used is CALPRENE sold by Dynasol Group, Houston, Tex. By definition, block copolymers are composed of two mer units, and clusters of the identical mers form blocks along the polymer chain. As shown in FIG. 2A, styrenic block copolymers generally designated 20 that form triblock thermoplastic elastomers can include chains of soft, elastomeric butadiene segments 22 flanked by hard, rigid, thermoplastic styrene blocks 24. In use as shown in FIG. 2B, the rigid styrenic blocks 24 (see FIG. 2A) at the ends of the chain 20 introduce the thermoplastic properties of the material and aggregate to form physical, i.e., non-covalent, cross-links 26. These cross-links 26 can be melted and reformed repeatedly and are therefore described as recyclable.

As stated above, the oily substance used in the bolus material 10 can be a mineral oil, for example, a clearly transparent, or water-clear, white mineral oil to solvate the block copolymer. The mineral oil can be a heavy mineral oil or a light mineral oil. The oil serves as a solvent for the styrenic block copolymers, which when heated dissolves and when cooled forms cross-links within the oil, producing an elastomeric gel. Typically, oil can comprise about 75% to about 92% by weight of the mixture in an oil gel blend. Non-limiting examples of white mineral oils that can be used include KAYDOL manufactured and sold by Sonneborn, Inc., of Tarrytown, N.Y., DRAKEOL manufactured and sold by Penreco of Dickinson, Tex., CRYSTAL PLUS sold by STE Oil Company of San Marcos, Tex. and CLARION sold by CITGO Petroleum Products of Houston, Tex. All these non-limiting examples of white mineral oil have been FDA approved for human skin contact.

Alternatively, as stated above, naphthenic oils such as TUFFLO 6000 Series Oils manufactured by CITGO Petroleum Corporation of Houston, Tex., can be used as the oily substance in the oil gel. Similarly, oligomers can be used as the oily substance in the oil gel. For example, polyterpene oligomers such as PICCOLYTE products manufactured by Hercules, Incorporated, of Wilmington, Del., and polybutene oligomers such as INDOPOL H300 manufactured by Natrochem, Inc., of Savannah, Ga., can be used as the oily substance in the oil gel.

In an embodiment where mineral oil is used as the oily substance in the oil gel, bolus material 10 can comprise about 8% to about 25% by weight of a styrenic block copolymer or copolymers and about 75% to about 92% by weight of a mineral oil. Non-limiting examples of the styrenic block copolymers that can be used in conjunction with mineral oil include KRATON G polymers such as KRATON G1650M polymer, KRATON G1651H polymer, and KRATON G1652M polymer. For example, between about 10% and about 15% of KRATON G1650M polymer can be used in the formation of bolus material 10. In other example embodiments, about 8% of KRATON G1651H polymer can be used as the styrenic block copolymer. In further example embodiments, between about 10% and about 20% of KRATON GS 652M polymer can be used as the styrenic block copolymer. Other various amounts and percentages of KRATON G1650M polymer, KRATON G1651H polymer, and KRATON G1652M polymer can be used. In some embodiments, different combinations of KRATON G1650M polymer, KRATON G1651H polymer, and KRATON G1652M polymer can be used.

In one embodiment, the bolus material can include about 15% KRATON G1652M polymer and about 85% white mineral oil. This bolus material is clear and has a low Young's Modulus of 0.039 GPa. This bolus material also is soft to the touch due to a Shore A hardness of 69 for the KRATON G1652M polymer. Maximum strain for such a bolus material is about 62%. The KRATON G1652M polymer also ensures stability and has also been approved by the FDA. The dose characteristics are within about 5% to about 10% of desired value, which can be adequately compensated. Finally, the material has satisfactory tackiness rating based on qualitative observation.

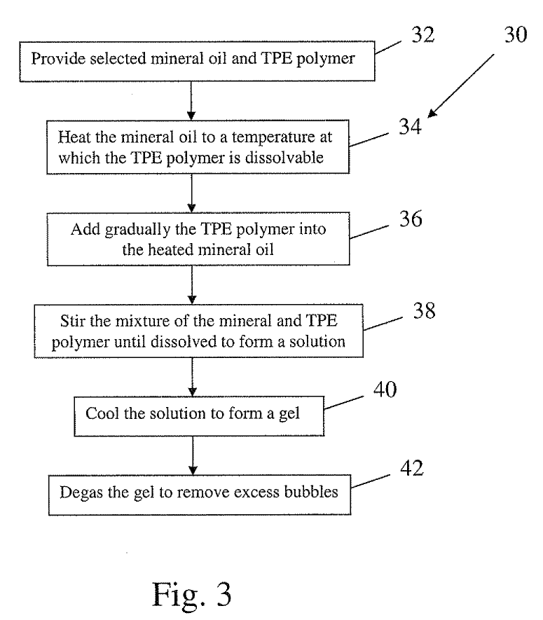

An embodiment of a method 30 of producing a bolus material is provided below in FIG. 3. An appropriate amount of mineral oil and TPE polymer are provided in step 32. The mineral oil is heated in step 34 to a temperature at which the TPE polymer to be used will dissolve in the mineral oil. Once appropriately heated, the TPE polymer can be dissolved in the mineral oil. In step 36, the TPE polymer can be gradually added and the mixture stirred until the TPE polymer is dissolved to form a solution. In step 38, the solution can then be cooled to form a gel. For example, the mineral oil can be heated to a temperature of between about 100° C. and about 150° C., for instance about 130° C. The TPE polymer in the form of a KRATON polymer can be gradually added. The mixture can be stirred at this temperature for approximately 1 to 2 hours, until the polymer is dissolved. The solution can then be cooled to form a gel that is processable to be clearly transparent and provides the characteristics of a maximum strain of at least 50%, a Young's Modulus of less than 0.1 GPa, and a hardness less than about 90 on the Shore A scale. The gel can be formed, for example, on a TEFLON coated tray or into a mold that allows the gel to form at a desired thickness and shape.

Optionally, with step 40, an additional degassing step 42 can be included to remove excess bubbles from the gel. For example, the gel can be heated in a vacuum oven to a temperature of between about 100° C. and about 150° C., for instance about 130° C., in about −25 mmHg pressure for about 1 hour until bubbles are removed. The gel can remain in a mold that allows the gel to reform at a desired thickness and shape. The resulting gel can then be used as a bolus material.

Bolus material 10 can have a Young's modulus in the range of about 35 MPa to about 122 MPa. Increasing the styrenic block copolymer(s) concentration generally creates a less flexible and stiffer material. This result occurs since increasing the styrenic block copolymer(s) concentration requires replacing oil molecules with higher-weight styrenic components. A higher molecular weight polymer will generally produce a higher Young's Modulus for a given concentration.

Bolus material 10 can generally have a tensile strength modulus in the range of about 20 MPa to about 100 MPa. The tensile strength increases as polymer concentration increases. Generally, this increase in tensile strength will result from increased crosslink formation. It should be emphasized that the low tensile strength is not a major disadvantage. The bolus is not intended for use in rigorous environments, and the strength is adequate if the bolus material does not fall apart upon handling. Therefore, for the purposes of radiation therapy, the tensile strength of about 20 MPa to about 50 MPa for the bolus material is sufficient. However, the tensile strength can be greater than about 50 MPa. Furthermore, Young's Modulus appears to increase with weight percentage polymer at a much faster rate than tensile strength. Increasing the tensile strength by increasing polymer concentration would entail sacrificing softness and drape by increasing the Young's Modulus. As long as the bolus material can hold together, the drape properties of the bolus material are more important than increased strength to provide more accurate dosage during radiation therapy.

Bolus material 10 can have an ultimate elongation or maximum strain of about 50% to about 165%. Again, drape properties should not be sacrificed to obtain greater elongation. A maximum strain of about 50% of the bolus material is sufficient. Similarly, depending on the TPE polymers used, tackiness can generally decrease with increasing polymer percentage, and decreasing tackiness levels can correlate with decreased drapeability. While the bolus material should not be too tacky, the elimination of tackiness from the bolus material generally should not sacrifice drapeability. As explained in more detail below, tackiness of the oil gels can be measured by contact angle. For example, a contact angle of less than about 160° can be sufficient.

Bolus materials of oil gel that includes styrenic block copolymer(s) and mineral oil generally are clearly transparent. The transparency is generally clear enough to observe minute features on the skin surface of the patient and markings on the patient through the bolus material. Such clarity of material provides a benefit to the radiation oncologist who uses the bolus material for radiation therapy of lesion on the skin surface.

The dosimetric properties of the bolus materials of an oil gel that includes the styrenic block copolymer(s) and mineral oil can deviate about 5% to about 10% from the desired values as compared to tissue-equivalent dosimetric properties. Again, because the density of the bolus materials can be ultimately less than 1 g/cm³, the materials can have less than tissue-equivalent absorption. Thus, the bolus materials can be slightly thicker than the equivalent thickness of tissue, for example, about 5% to about 10% thicker. For example, for the equivalence of 1 cm of tissue, the bolus materials can be slightly thicker than 1 cm, for example about 1.05 to about 1.1 cm. The increase in thickness can help ensure dose build-up on the skin surface.

The bolus materials of an oil gel that includes styrenic block copolymer(s) and mineral oil is nontoxic and provides a low environmental impact. The bolus material can be reused as long as it is kept clean, contributing to decreased wasted generation. In addition, since the bolus materials of a thermally-stable, thermoplastic oil gel that includes styrenic block copolymer(s) and mineral oil is comprised mostly of mineral oil, it can be repeatedly recycled into new bolus sheet by reheating and reprocessing.

Therefore, the bolus materials of oil gel that includes styrenic block copolymer(s) and mineral oil provide a safe, transparent, odorless, soft, and drapeable material that is strong enough to be used by radiation oncologists in radiation therapy to concentrate the ionizing radiation on the skin surface on which the bolus material is placed. Such a bolus material enables more accurate treatment during radiation therapy.

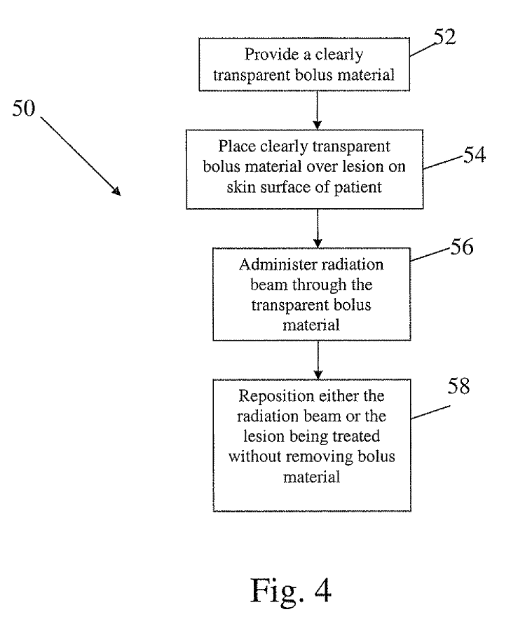

Such bolus material can be beneficial for use in radiation therapy. By using such bolus material, the radiation oncologist can clearly see the lesion to be treated on the skin surface of the patient through the bolus material. A method generally designated 50 for using the bolus material is provided in FIG. 4. Method 50 includes a step 52 of providing a clearly transparent bolus material of oil gel comprising at least one thermoplastic elastomer and a mineral oil. As described above, the mineral oil can be a white mineral oil and the thermoplastic elastomer can be any suitable styrenic block copolymer such as second generation styrenic block copolymers with a hydrogenated midblock of styrene-ethylenelbutylene-styrene or styrene-ethylene/propylene-styrene. An example includes a linear triblock copolymer based on styrene and ethylene/butylene with a Styrene/Rubber ratio of 30/70.

In step 547 the clearly transparent bolus material can be placed over a lesion on a skin surface of a patient such that the lesion is visible through the bolus material. The bolus material being used in the radiation therapy should be of a thickness that permits the radiation dose within the lesion to be close to maximum while the target lesion is still clearly visible beneath the bolus. Due to the fact that the bolus material is clearly transparent, the radiation oncologist can position the lesion to be treated by visual inspection in a location where the radiation beams can be focused thereon, even after the bolus material is placed over the area of the skin surface where the lesion is. Alternatively, the beams can be positioned by visual inspection of the lesion through the clearly transparent bolus, so that the radiation beams are focused on the target lesion. The clearly transparent bolus material also permits the radiation oncologist to clearly see guide markings placed on the patient as well as minute details of the lesion to be treated and the surrounding skin surface covered by the bolus material.

In step 56, radiation beams can be administered through the transparent bolus material such that dosage of radiation is increased on the lesion at the skin surface of the patient. Optimally, the radiation dosage is maximized within the lesion to be treated generally at the skin surface. The patient can be positioned and aligned to receive the radiation beams with the bolus material in place, since the radiation oncologist can see the target area through the bolus material. The thickness of the bolus material can be generally determined before use to maximize the radiation dosage level throughout the thickness of the lesion.

Optionally, due to the transparency of the bolus material, the radiation oncologist can reposition either the radiation beam or the lesion on the skin surface of the patient without removing the bolus material in step 58. Thus, being able to see through the bolus material can increase the accuracy of alignment, the speed of realignment and the overall speed of treatment.

The use of a clear bolus material will improve the precision and accuracy of radiation therapy treatment. For example, by being able to clearly see the target, the radiation oncologist can precisely and accurately adjust the position of the patient or the radiation beam such that the target is completely irradiated and the surrounding normal tissue is not. As a result, the radiation oncologist reduces the risk of missing the tumor. In addition, by reducing the uncertainty about the lesion location, the radiation oncologist can reduce the margin of normal tissue that the radiation oncologist would normally include in the radiation field to ensure that the radiation treatment does not miss the lesion.

In the above described manner, the bolus material described above can beneficially improve radiation therapy treatment of lesions or other malformations that occur on or near the skin surface of a patient.

### EXAMPLES

Bolus materials of an oil gel that includes styrenic block copolymer(s) and mineral oil were created that were tested to determine tensile, tackiness, transparency and dosimetric properties. The styrenic block copolymers used were three KRATON polymers for inclusion in the oil gel: KRATON G1650M, G1651H, and G1652M polymers. The G1652M polymer has a low molecular weight, low viscosity, and relatively low tear strength. The G1650M polymer has a medium molecular weight, and moderate viscosity and tear strength. The G1651H polymer has a high molecular weight, a higher styrene to rubber ratio, a very high viscosity, and high tear strength. These properties are summarized in Table 2.

TABLE 2
Comparison of three KRATON G polymers

| | KRATON | KRATON | KRATON |
|---|---|---|---|
| Property | G1652 | G1650 | G1651 |
| Relative MW | low | medium | high |
| Styrene/ | 29/71 | 29/71 | 32/68 |
| rubber ratio | | | |
| Physical form | powder | powder | powder |
| FDA approved? | Yes | yes | yes |
| Viscosity (cP), | 12 | 18 | 42700 |
| 5% wt | | | |
| Flow at 50 | >100% | <100% | no flow |
| degrees for | deformation | deformation | after 16 hours |
| 5% weight | after 16 hrs | after 16 hours | |
| Tear strength, J/m | 21 | 75 | 475 |
| Shore A hardness | 69 | 72 | 60 |

### I. Preparation of the Bolus Materials

For each different embodiment of the bolus materials, approximately 300 mL of oil was heated to a temperature of 130° C. and different amounts of the KRATON G1650M, G1651H, and G1652M polymers were gradually added, respectively. Each mixture was stirred at this temperature for approximately 1-2 hours, until the respective polymer dissolved. The solution was poured onto a baking sheet and cooled to form a gel. Later, the gel was heated in a vacuum oven at 130° C. in −25 mmHg pressure until bubbles were removed, about 1 hour.

### II. Testing: Measurement of Tensile Properties

The tensile properties of the bolus materials were determined by calculating their maximum strain, tensile stress, and Young's modulus. To obtain these values, 1-inch wide sections of bolus material were cut and attached to a Tinius-Olson rheometer. The initial length and cross-sectional area were measured. The tensile force was measured until fracture and the force and extension data were converted to stress in GPa by the equation:

σ=F/A

Strain is unitless and was obtained by the equation:

ε=ΔL/L₀

The maximum strain was calculated by taking the largest extension of the bolus material before fracture and dividing this value by the original length. The tensile stress was calculated by taking the stress of the material at fracture. To calculate Young's Modulus, the slope of the stress-strain curve was measured at “small” strain values, according to the equation:

E=σ/ε

which provides a result in GPa. “Small” strain refers to lengths in which there is linear response in the bolus material and occurs in most case before the strain equals 0.1. The low value was calculated at small strains (below 0.5 strain) and the high value was calculated when the stress-strain curve was linear (at strains ranging from 1 to 1.5).

### III. Testing: Contact Angle Measurement

In order to quantify tackiness, contact angle was measured. The contact angle is defined as the angle made by a drop of liquid on a material where the edge of the droplet contacts the underlying surface. The contact angle is measured by doubling the angle between the horizontal and the line connecting the contact point and the apex of the droplet.

In general, liquids will spread if the surface tension of the liquid is lower than the critical surface tension of the surface. The lower the interfacial free energy of the surface, the more likely the liquid is to not interact with the surface. The liquid used for the measurements was water. Since the tackier materials were considered to have more oil on the surface, and oil and water do not mix, tackier materials should have caused the water bead to ball up more, leading to a higher contact angle.

A CAM-MICRO contact angle meter was used to obtain the half-angle of each specimen. Each sample was placed onto the contact angle meter, positioned properly, and a droplet of water was applied to each surface. The half-angle was measured and was multiplied by 2 to find the contact angle.

### IV. Transparency and Dosimetric Testing

The transparency was measured by a simple pass-fail test. A 1-cm slab of the novel material was placed on a sheet of 12-point newsprint and if the print could be read through the material, it was considered sufficiently transparent.

To quantify the dosimetric properties, the client used x-ray attenuation in a CT scanner to measure CT number, electron density, and effective atomic number Z.

The materials were placed in a CT scanner, and the linear attenuation coefficient μwas measured. To determine the CT number, μ for the material was normalized with μ_{water} according to the equation

C=1000*((μ−μ_{water})/μ_{water})

Next, the effective atomic number Z, or average number of electrons per atom, was approximately a function of two attenuation measurements, μ₁ and μ₂ according to the equation

Z=μ₁/μ₂

Finally, the electron density ρ in e⁻/cm³ was also a function of two attenuation measurements, by

ρ=μ₁−kμ₂

where k is a constant.

### V. Different Bolus Materials Tested

For use in radiation therapy, the bolus material should have a formulation that provides a balance of softness, strength, low tack, and transparency. An initial formulation of 8% KRATON G1652 polymer was created. A total of eleven boluses by varying concentration and type of KRATON polymer were created. Seven of these boluses (A-G) were ultimately tested to quantify the material properties. The materials were created in the order shown in Table 3.

Changes in concentration and composition for subsequent gels were based on qualitative observations of drape, tack, and strength. Also, Table 2 was consulted frequently during the experiments. After the different bolus materials were formulated, quantitative tests of drape, tack, and strength were performed, along with transparency and dosimetric tests.

TABLE 3
Oil gel formulations

| | Bolus | | |
|---|---|---|---|
| ID | type | Qualitative Assessment | Conclusion |
| AA | 8.1% | low tear strength | use higher molecular weight |
| | G1652 | | (MW) polymer, increase polymer % |
| BB | 10% G1650 | tacky according to client, | increase polymer % |
| | | low density | |
| CC | 20% G1650 | Poor drape, stiff; not tacky, | add fumed silica filler, decrease |
| | | low density | polymer % |
| DD | gels with | inelastic gel formed, | omit fumed silica, implement |
| | filler | opaque (silica acted as | conversion factor for tissue- |
| | | gelling agent) | equivalent thickness |
| D | 13% G1650 | improved drape, tacky from | add high MW polymer, lower |
| | | client observation | overall % polymer |
| C | 10% | tacky | try low MW polymer of |
| | G1650, 1% | | comparable % for comparison |
| | G1651 | | of properties |
| B | 10% G1652 | tacky, weak | increase % polymer |
| G | 20% G1652 | somewhat stiff | decrease % polymer or try low % |
| | | | of high MW polymer |
| A | 8% G1651 | uneven texture | try high % of low MW polymer |
| E | 15% G1652 | good drape, good overall | increase % polymer to make fine |
| | | properties, slightly tacky | range |
| F | 17% G1652 | better tack, slightly worse | 15-17% G1652 meets required |
| | | drape than E | function |

The first three boluses in Table 3 were not degassed. For all the experimental boluses created, a white mineral oil was used. The first bolus was created with 8% KRATON G1652 and a generic white mineral oil. The next bolus was produced with the KRATON G1650M polymer, a polymer with higher tear strength. In the second gel, the concentration was increased to 10% to increase strength. The oil gel with 10% KRATON G1650M polymer showed improvement over the initial bolus. The next gel produced contained 20% KRATON G1650M polymer. This gel was perceived to be a very viscous gel that trapped many bubbles upon cooling.

Experiments with silica filler were performed and methods for degassing were investigated. The silica filler used was CAB-O-SIL M5P manufactured and sold by Cabot Corporation of Billerica, Mass. The use of silica filler caused the mixture to become extremely viscous and putty-like, such that a solid gel did not form upon cooling. Also, the silica filler interfered with the transparency of the product.

The next gel made included 13% KRATON G1650M polymer. The gel was perceived to be somewhat less tacky but adequate drape was maintained. Another product with 10% KRATON G1650M polymer and 1% KRATON G1651H polymer was produced. A gel of 10% KRATON G1652M polymer was also created, in order to compare two gels of the similar concentration with a different mix of polymers. Both of these gels were perceived to be somewhat tackier than the gel that included 13% KRATON G1650M polymer.

Using a vacuum oven, bubbles were able to be removed that were trapped in the gels. Starting with the gel including 13% KRATON G1650M polymer, all the gels made previously were successfully degassed and degassing was included as a final step in the mixing protocol.

A gel that included 20% KRATON G1652M polymer was then produced. Since the 20% KRATON G1652M polymer was perceived to have more limited flexibility but favorable tack properties, the final gels produced included 15% KRATON G1652M polymer and 17% KRATON G1652M polymer.

Bolus materials A-G in Table 3 were tested with the quantitative experiments described above. The results of these tests appear in the following section.

### Experimental Results

The results of tests to characterize the bolus materials appear below.

### I. Mechanical Properties

The maximum strain, tensile stress, and Young's Modulus for the tested boluses are listed in table 4.

TABLE 4
Mechanical Properties of Candidate Materials

| | Bolus | Max Strain | Tensile Stress | Young's |
|---|---|---|---|---|
| ID | Type | (Unitless) | (GPa) | Mod (GPa) |
| A | 8% 1651 | 3.5 | 0.022 @ 3.5 Strain | 0.015 |
| | | (No Fracture) | (No Fracture) | |
| B | 10% 1652 | 0.21 | 0.0027 | 0.021 |
| C | 10% 1650, | 1.2 | 0.026 | 0.052 |
| | 1% 1651 | | | |
| D | 13% 1650 | 1.2 | 0.029 | 0.071 |
| E | 15% 1652 | 0.62 | 0.013 | 0.039 |
| F | 17% 1652 | 0.65 | 0.020 | 0.066 |
| G | 20% 1652 | 0.75 | 0.038 | 0.12 |

### II. Contact Angle

The results of the contact angle measurements appear in Table 5.

TABLE 5
Contact angle results

| | Polymer | |
|---|---|---|
| ID | Composition | Contact Angle |
| A | 8% 1651 | Not tested |
| B | 10% 1652 | 140° |
| C | 10% 1650, | Not tested |
| | 1% 1651 | |
| D | 13% 1650 | 156° |
| E | 15% 1652 | 148° |
| | | (interpolated) |
| F | 17% 1652 | 152° |
| G | 20% 1652 | 156° |

Polymers A, C, and E were not tested. Interpolation was used to estimate Polymer E.

### III. Transparency and Dosimetric Results

Table 6 displays the results of dosimetric tests. Concerning the testing of transparency, all of the tested boluses exhibited sufficient transparency. Each of the tested boluses was transparent enough to permit typewritten writing in 12 point font to be read through the tested bolus having a thickness of 1 cm.

TABLE 6
Dosimetric results

| | Novel Boluses | Water/Tissue |
|---|---|---|
| Transparency | Can read 12 point newsprint | — |
| CT number | 130-160 HU | 90 HU |
| Mean Z | 5.4 | 7.42 |
| Electron Density | 3.05 × 10²³ e⁻/cm³ | 3.34 × 10²³ e⁻/cm³ |

Embodiments of the present disclosure shown in the drawings and described above are exemplary of numerous embodiments that can be made within the scope of the appending claims. It is contemplated that the configurations described herein can comprise numerous configurations other than those specifically disclosed. The scope of a patent issuing from this disclosure will be defined by these appending claims.

What is claimed is:

1. A bolus material for use in radiation therapy comprising a oil gel comprising at least one thermoplastic elastomer and a mineral oil with the at least one thermoplastic elastomer comprising between about 8% and about 25% of the oil gel and the mineral oil comprising between about 75% and about 92% of the oil gel whereby the oil gel is clearly transparent and has a maximum strain of at least 50%, a Young's Modulus of less than about 0.1 GPa, and a hardness less than about 90 on the Shore A scale.

2. A bolus material according to claim 1, wherein the at least one thermoplastic elastomer comprises at least one styrenic block copolymer.

3. A bolus material according to claim 2, wherein the at least one styrenic block copolymer comprises at least one second generation styrenic block copolymer with a hydrogenated midblock of at least one of a styrene-ethylene/butylene-styrene or a styrene-ethylene/propylene-styrene.

4. A bolus material according to claim 1, wherein the at least one thermoplastic elastomer comprises a linear triblock copolymer based on styrene and ethylene/butylene with a Styrene/Rubber ratio of 30/70 that comprises between about 15% and about 17% of the oil gel.

5. A bolus material according to claim 1, wherein the mineral oil comprises a white mineral oil.

6. A bolus material according to claim 1, wherein the oil gel is odorless.

7. A bolus material according to claim 1, wherein the bolus material has a computed tomography number between about 130 HU to about 160 HU, a calculated mean atomic number Z of about 5.4 and electron density of about 3.05×10²³ e⁻/cm.

8. A bolus material according to claim 7, wherein the bolus material comprises a thickness that is about 5% to about 10% larger than a thickness of its soft tissue equivalence for dosimetric properties.

9. A bolus material according to claim 1, wherein the bolus material is sufficiently transparent that a person having 20/20 vision can read black typing in 12 point font through at least 1 cm of the bolus material.

10. A bolus material for use in radiation therapy comprising a oil gel comprising at least one thermoplastic elastomer and an oily substance, whereby the oil gel is clearly transparent and has a maximum strain of at least 50%, a Young's Modulus of less than about 0.1 GPa, and a hardness less than about 90 on the Shore A scale, the bolus material being configured to increase a radiation dosage being administered to a lesion on or near a skin surface of a patient while permitting the lesion to be visible through the bolus material.

11. A bolus material according to claim 10, wherein the oily substance comprises at least one of a mineral oil, a naphthenic oil, a polyterpene oligomer or polybutene oligomer.

12. A bolus material according to claim 10, wherein the at least one thermoplastic elastomer comprises at least one styrenic block copolymer.

13. A bolus material according to claim 12, wherein the at least one styrenic block copolymer comprises at least one second generation styrenic block copolymer with a hydrogenated midblock of at least one of a styrene-ethylene/butylene-styrene or a styrene-ethylenelpropylene-styrene.

14. A bolus material according to claim 10, wherein the oil gel is odorless.

15. A bolus material according to claim 10, wherein the bolus material is sufficiently transparent that a person having 20/20 vision can read black typing in 12 point font through at least 1 cm of the bolus material.

16. The method of providing radiation therapy, the method comprising:
providing a clearly transparent bolus material;
placing the clearly transparent bolus material over a lesion on or near a skin surface of a patient such that the lesion is visible through the bolus material;
administering radiation beams through the clearly transparent bolus material such that dosage of radiation is increased on the lesion on or near the skin surface.

17. The method according to claim 16, wherein the bolus material comprises an oil gel including at least one thermoplastic elastomer and an oily substance with the oil gel providing the characteristics of being clearly transparent, a maximum strain of at least 50%, a Young's Modulus of less than about 0.1 GPa, and a hardness less than about 90 on the Shore A scale.

18. A bolus material according to claim 17, wherein the oily substance comprises a mineral oil.

19. The method according to claim 18, wherein the at least one thermoplastic elastomer comprises between about 8% and about 25% of the oil gel and the mineral oil comprising between about 75% and about 92% of the oil gel.

20. The method according to claim 18, wherein the at least one thermoplastic elastomer comprises at least one styrenic block copolymer.

21. The method according to claim 20, wherein the at least one styrenic block copolymer comprises at least one second generation styrenic block copolymer with a hydrogenated midblock of at least one of a styrene-ethylene/butylene-styrene or a styrene-ethylene/propylene-styrene.

22. A bolus material according to claim 17, wherein the oily substance comprises at least one of a mineral oil, a naphthenic oil, a polyterpene oligomer or polybutene oligomer.

23. The method according to claim 16, further comprising repositioning at least one of the radiation beam or the lesion on or near the skin surface of the patient without removing the transparent bolus material.

24. A bolus material according to claim 16, wherein the bolus material is sufficiently transparent that a person having 20/20 vision can read black typing in 12 point font through at least 1 cm of the bolus material.
